(19) United States
(12) Patent Application Publication
(10) Pub. No.: US 2025/0282875 A1
(43) Pub. Date: Sep. 11, 2025

(54) METHODS OF TREATING COLORECTAL CANCER USING AN ANTI-CTLA4 ANTIBODY

(71) Applicant: AGENUS INC., Lexington, MA (US)

(72) Inventors: Dhan Sidhartha CHAND, Lexington, MA (US); Steven O'DAY, Lexington, MA (US); Joseph GROSSMAN, Lexington, MA (US)

(73) Assignee: AGENUS INC., Lexington, MA (US)

(21) Appl. No.: 19/216,370

(22) Filed: May 22, 2025

Related U.S. Application Data
(63) Continuation of application No. 19/126,151, filed on Jan. 1, 0001.
(60) Provisional application No. 63/381,621, filed on Oct. 31, 2022.
(60) Provisional application No. 63/497,392, filed on Apr. 20, 2023.

(51) Int. Cl. C07K 16/28; A61K 39/00; A61P 35/04

(52) U.S. Cl. CPC C07K 16/2818; A61P 35/04; A61K 2039/507; A61K 2039/54; A61K 2039/545; C07K 2317/565

(57) ABSTRACT

Provided are methods for treating colorectal cancer with an antibody that specifically binds to human Cytotoxic T-Lymphocyte Antigen 4 (CTLA-4).

### RELATED APPLICATIONS

This application is a continuation of U.S. patent application Ser. No. 19/126,151, filed Apr. 30, 2025, which is a U.S. National Phase Application under 35 U.S.C. § 371 of International Application No. PCT/US2023/036433, filed Oct. 31, 2023, which claims priority to U.S. Provisional Patent App. No. 63/381,621, filed Oct. 31, 2022, and to U.S. Provisional Patent App. No. 63/497,392, filed Apr. 20, 2023, the entire disclosure of each of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Oct. 24, 2023, is named “205370_seglist.xml” and is 19,846 bytes in size).

### BACKGROUND

Colorectal cancer (CRC) is one of the most common cancers in the world and one of the leading causes of cancer-related mortality. In the setting of metastatic CRC (mCRC), the 5-year survival rate is less than 20% for those with distant metastases.

Currently, the standard initial treatment for patients with mCRC is systemic combination chemotherapy, with agents that have been in use for decades (e.g., 5-fluorouracil, oxaliplatin, irinotecan). More recent initial treatment options used in combination with chemotherapy include vascular endothelial growth factor (VEGF) therapy (e.g., bevacizumab), and epidermal growth factor receptor (EGFR) targeting therapy (cetuximab or panitumumab). However, these treatments are only modestly effective. Further, although immunotherapy approaches have shown success in various types of cancer, including high microsatellite instability/mismatch repair deficient (MSI-H/dMMR) mCRC, immunotherapy has not yet shown success for treating non-MSI-H/dMMR mCRC. In the mCRC population, greater than 95% of patients are non-MSI-H/dMMR, and approximately 30-35% of these patients, including those participating in clinical studies, do not have liver metastases.

The vast majority of patients with CRC (including those without liver metastases) are ineligible for immune checkpoint inhibitors as standard of care due to non-MSI-H/dMMR status, and this patient population relies on modestly efficacious chemotherapy and targeted therapy regimens, making it an area of significant unmet medical need.

Accordingly, there remains a need for novel and efficacious methods of treating metastatic colorectal cancer.

### SUMMARY

The instant disclosure is directed to methods for treating colorectal cancer with an antibody that specifically binds to human Cytotoxic T-Lymphocyte Antigen 4 (CTLA-4). Also provided herein are particular methods for administering an antibody that specifically binds to human CTLA-4 that results in a reduction of tumor burden in a subject. In an embodiment, the method comprises a therapeutically effective amount that safely and effectively treats metastatic colorectal cancer. In an embodiment, the method comprises a therapeutically effective amount that safely and effectively reduces the tumor burden in a subject that has metastatic colorectal adenocarcinoma.

In an aspect, provided herein is a method of treating colorectal cancer in a subject in need thereof, the method comprising administering to the subject an antibody that specifically binds to human Cytotoxic T-Lymphocyte Antigen 4 (CTLA-4) at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has colorectal cancer, the method comprising administering to the subject 25 mg to 200 mg of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody is administered at a dose of 50 mg to 175 mg. In an embodiment, the antibody is administered at a dose of 75 mg to 150 mg. In an embodiment, the antibody is administered at a dose of about 25 mg, 50 mg, 75 mg, 100 mg, or 150 mg. In an embodiment, the antibody is administered at a dose of 25 mg, 50 mg, 75 mg, 100 mg, or 150 mg.

In an embodiment, the antibody is administered intravenously. In an embodiment, the antibody is administered by intravenous infusion over about 30 minutes.

In an embodiment, the antibody is administered once weekly. In an embodiment, the antibody is administered once every 2 weeks. In an embodiment, the antibody is administered once every 3 weeks. In an embodiment, the antibody is administered once every 4 weeks. In an embodiment, the antibody is administered once every 5 weeks. In an embodiment, the antibody is administered once every 6 weeks.

In an embodiment, the antibody is administered intravenously at a dose of 25 mg once every 6 weeks. In an embodiment, the antibody is administered intravenously at a dose of 50 mg once every 6 weeks. In an embodiment, the antibody is administered intravenously at a dose of 75 mg once every 6 weeks. In an embodiment, the antibody is administered intravenously at a dose of 100 mg once every 6 weeks. In an embodiment, the antibody is administered intravenously at a dose of 150 mg once every 6 weeks.

In an embodiment, the dose is a therapeutically effective amount.

In an embodiment, the colorectal cancer is colorectal adenocarcinoma. In an embodiment, the colorectal cancer is unresectable. In an embodiment, the colorectal cancer is metastatic. In an embodiment, the colorectal cancer is metastatic, unresectable colorectal adenocarcinoma. In an embodiment, the subject does not have liver metastases.

In an embodiment, the antibody is administered to the subject prior to surgical resection of a primary tumor. In an embodiment, the subject has not received any prior chemotherapy. In an embodiment, the subject has not received any prior radiation therapy.

In an embodiment, the colorectal cancer is relapsed and/or refractory.

In an embodiment, the subject has received at least one prior chemotherapy. In an embodiment, the at least one prior chemotherapy is fluoropyrimidine, irinotecan, oxaliplatin, or an anti-EGFR antibody. In an embodiment, the anti-EGFR antibody is cetuximab or panitumumab.

In an embodiment, the subject has previously been treated with folinic acid, 5-fluorouracil, oxaliplatin, and/or irinotecan. In an embodiment, the subject is unable to tolerate a standard of care treatment.

In an embodiment, the subject has a RAS mutation. In an embodiment, the RAS mutation is a KRAS or NRAS mutation.

In an embodiment, the colorectal cancer is not microsatellite instable—high (MSI-H). In an embodiment, the colorectal cancer is microsatellite stable (MSS). In an embodiment, the colorectal cancer is not mismatch repair deficient (dMMR).

In an embodiment, the subject has not previously been treated with anti-PD-1, anti-PD-L1, or anti-CTLA-4 antibody. In an embodiment, the subject has not received prior regorafenib, trifluridine, and/or tipiracil therapy.

In an embodiment, the cancer is refractory to a standard of care treatment. In an embodiment, the standard of care treatment is chemotherapy or radiation. In an embodiment, the standard of care treatment is folinic acid, 5-fluorouracil, oxaliplatin, irinotecan, fluoropyrimidine, cetuximab, and/or panitumumab.

In an embodiment, the administration of the antibody reduces tumor size in the subject. In an embodiment, the administration of the antibody increases T-cell, memory T cell, myeloid cell, and/or antigen presenting cell activation in the subject. In an embodiment, administration of the antibody reduces the number of Treg cells in the subject.

In an embodiment, before administration of the antibody the subject has measurable disease on baseline imaging per RECIST 1.1. In an embodiment, before administration of the antibody the subject has an Eastern Cooperative Oncology Group performance status (PS) 0-1. In an embodiment, before administration of the antibody the subject has a predicted life expectancy of ≥12 weeks.

In an embodiment, before administration of the antibody the subject has: adequate organ function as defined by one or more of: a) neutrophils≥1500/μL; b) platelets≥100×103/μL; c) hemoglobin≥8.0 g/dL; d) creatinine clearance≥30 mL/min as measured or calculated per local institutional standards; e) AST/ALT≤2.5× upper limit of normal (ULN); f) total bilirubin≤1.5×ULN (except patients with Gilbert syndrome who must have a total bilirubin level of ≤3.0×ULN); and/or g) albumin≥3.0 g/dL.

In an embodiment, the subject does not have partial or complete bowel obstruction within the last 3 months, signs/symptoms of bowel obstruction, or known radiologic evidence of impending obstruction.

In an embodiment, the subject does not have refractory ascites defined as requiring 2 or more therapeutic paracenteses within the last 4 weeks or ≥4 times within the last 90 days or ≥1 time within the last 2 weeks prior to administration of the antibody.

In an embodiment, the subject does not have clinically significant cardiovascular disease.

In an embodiment, the subject does not have active brain metastases or leptomeningeal metastases. In an embodiment, the subject does not have a concurrent malignancy that requires treatment or a history of prior malignancy that was active within 2 years prior to administration of the antibody.

In an embodiment, the subject has not had cytotoxic therapy, targeted therapy, or other investigational therapy within 3 weeks prior to administration of the antibody. In an embodiment, the subject has not had other monoclonal antibody, antibody-drug conjugate, or radioimmunoconjugate therapy within 4 weeks prior to administration of the antibody. In an embodiment, the subject has not had small molecule tyrosine kinase inhibitor therapy within 2 weeks prior to administration of the antibody.

In an embodiment, the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a human IgG1 heavy chain constant region comprising S239D/A330L/I332E mutations, numbered according to the EU numbering system.

In an embodiment, the antibody comprises a heavy chain comprising the amino acid sequence set forth in SEQ ID NO: 9 and a light chain comprising the amino acid sequence set forth in SEQ ID NO: 10.

In an embodiment, the antibody is botensilimab.

In an aspect, provided herein is a method of treating metastatic colorectal adenocarcinoma in a subject in need thereof, the method comprising administering to the subject an antibody that specifically binds to human CTLA-4 at a dose of 75 mg or 150 mg once every 6 weeks, wherein the antibody is botensilimab.

In an embodiment, the metastatic colorectal adenocarcinoma is unresectable. In an embodiment, the metastatic colorectal adenocarcinoma is non-MSI-H/dMMR.

In an embodiment, the method further comprises administering an antibody that specifically binds to human PD-1 to the subject.

In an embodiment, the antibody that specifically binds to human PD-1 comprises: a heavy chain variable region (VH) comprising the CDRH1, CDRH2, and CDRH3 amino acid sequences of the VH amino acid sequence set forth in SEQ ID NO: 17; and a light chain variable region (VL) comprising the CDRL1, CDRL2, and CDRL3 amino acid sequences of the VL amino acid sequence set forth in SEQ ID NO: 18.

In an embodiment, the antibody that specifically binds to human PD-1 comprises the CDRH1, CDRH2, CDRH3, CDRL1, CDRL2, and CDRL3 amino acid sequences set forth in SEQ ID NOs: 11, 12, 13, 14, 15, and 16, respectively.

In an embodiment, the antibody that specifically binds to human PD-1 comprises a VH comprising the amino acid sequence set forth in SEQ ID NO: 17 and a VL comprising the amino acid sequence set forth in SEQ ID NO: 18. In an embodiment, the antibody that specifically binds to human PD-1 comprises a heavy chain comprising the amino acid sequence set forth in SEQ ID NO: 19 and a light chain comprising the amino acid sequence set forth in SEQ ID NO: 20.

In an embodiment, the antibody that specifically binds to human PD-1 is balstilimab.

In an embodiment, the antibody that specifically binds to human PD-1 is administered at a dose of 200 mg to 300 mg. In an embodiment, the antibody that specifically binds to human PD-1 is administered at a dose of 240 mg.

In an embodiment, the antibody that specifically binds to human PD-1 is administered once weekly or once every 2 weeks. In an embodiment, the antibody that specifically binds to human PD-1 is administered at a dose of 240 mg once every 2 weeks.

In an aspect, provided herein is an antibody that specifically binds to human CTLA-4 for use in the treatment of colorectal cancer, wherein the treatment is performed according to the method of any one of the previous claims.

In an aspect, provided herein is an antibody that specifically binds to human CTLA-4 for use in the manufacture of a medicament for the treatment of colorectal cancer, wherein the treatment is performed according to the method of any one of the previous claims.

In an aspect, provided herein is a use of an antibody that specifically binds to human CTLA-4 for the treatment of colorectal cancer, wherein the treatment is performed according to the method of any one of the previous claims.

In an aspect, provided herein is an antibody that specifically binds to human CTLA-4 and an antibody that specifically binds to human PD-1 for use in the treatment of colorectal cancer, wherein the treatment is performed according to the method of any one of the previous claims.

In an aspect, provided herein is an antibody that specifically binds to human CTLA-4 and an antibody that specifically binds to human PD-1 for use in the manufacture of a medicament for the treatment of colorectal cancer, wherein the treatment is performed according to the method of any one of the previous claims.

In an aspect, provided herein is a use of an antibody that specifically binds to human CTLA-4 and an antibody that specifically binds to human PD-1 for the treatment of colorectal cancer, wherein the treatment is performed according to the method of any one of the previous claims.

### BRIEF DESCRIPTION OF THE DRAWINGS

FIG. 1 shows the percent change in sum of target lesions over time, starting at initiation of therapy, in an efficacy evaluable population group with no active liver metastases (n=70) treated with botensilimab (either 1 or 2 mg/kg every six weeks) and balstilimab (3 mg/kg every two weeks) over time, starting at initiation of combination/rescue therapy, according to aspects of the present disclosure.

FIG. 2 shows the best percent change in sum of target lesions in an efficacy evaluable population group with no active liver metastases (n=70) treated with botensilimab (either 1 or 2 mg/kg every six weeks) and balstilimab (3 mg/kg every two weeks), according to aspects of the present disclosure. Asterisks indicate patients showing a complete response (CR) or partial response (PR).

FIG. 3 shows the overall survival probability over time, starting at initiation of therapy, in an efficacy evaluable (EE) population group with no active liver metastases (n=70) and an intent to treat (ITT) population group with no active liver metastases (n=77) treated with botensilimab (either 1 or 2 mg/kg every six weeks) and balstilimab (3 mg/kg every two weeks), according to aspects of the present disclosure.

### DETAILED DESCRIPTION

The instant disclosure is directed to methods for treating colorectal cancer with an antibody that specifically binds to human Cytotoxic T-Lymphocyte Antigen 4 (CTLA-4). Also provided herein are particular methods for administering an antibody that specifically binds to human CTLA-4 that results in a reduction of tumor burden in a subject. In an embodiment, the method comprises a therapeutically effective amount that safely and effectively treats metastatic colorectal cancer. In an embodiment, the method comprises a therapeutically effective amount that safely and effectively reduces the tumor burden in a subject that has metastatic colorectal adenocarcinoma.

### Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In an embodiment, the antibody is a humanized monoclonal antibody. In an embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of Proteins of Immunological Interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other (see Table 1 below). In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of Proteins of Immunological Interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human CTLA-4). CDRH1, CDRH2, and CDRH3 denote the heavy chain CDRs, and CDRL1, CDRL2, and CDRL3 denote the light chain CDRs.

TABLE 1
CDR definitions

| | CDR Definitions | | |
|---|---|---|---|
| | Kabat | Chothia | MacCallum |
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (F), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen.

As used herein, the term “EU numbering system” refers to the EU numbering convention for the constant regions of an antibody, as described in Edelman G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “subject” includes any human or non-human animal. In an embodiment, the subject is a human.

As used herein, the term “effective amount” in the context of the administration of a therapy to a subject refers to the amount of a therapy that achieves a desired prophylactic or therapeutic effect.

As used herein, the term “standard of care” refers to the most common treatments prescribed for a particular type of cancer. In an embodiment, the standard of care for metastatic colon cancer includes fluorouracil, capecitabine, oxaliplatin, irinotecan, or trifluridine-tipiracil.

As used herein, the term “targeted therapy” refers to a therapy that inhibits a specific protein. In an embodiment, the targeted therapy inhibits a protein that is known to be important for growth and/or survival of colon cancer cells (e.g., EGFR).

As used herein, the term “cytotoxic therapy” refers to a therapy that blocks or slows cell division. In an embodiment, the cytotoxic therapy kills cancer cells. In an embodiment, the cytotoxic therapy is fluorouracil, capecitabine, oxaliplatin, irinotecan, or trifluridine-tipiracil.

As used herein, the term “tumor burden” refers to the number of cancer cells, the size of a tumor, or the amount of cancer in the body of the subject.

As used herein, the term “about” when referring to a measurable value, such as a dosage, encompasses variations of ±20%, ±15%, ±10%, ±5%, ±1%, or ±0.1% of a given value or range, as are appropriate to perform the methods disclosed herein.

### Anti-CTLA-4 Antibodies

Antibodies that specifically bind to human CTLA-4 (i.e., anti-CTLA-4 antibodies) that are useful in the methods and uses described herein include but are not limited to those listed below.

In an embodiment, the antibody comprises: a heavy chain variable region (VH) comprising the CDRH1, CDRH2, and CDRH3 amino acid sequences of the VH amino acid sequence set forth in SEQ ID NO: 7. In an embodiment, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, and CDRH3 amino acid sequences set forth in SEQ ID NO: 1, 2, and 3, respectively. In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7. In an embodiment, the antibody comprises: a VH comprising the amino acid sequence set forth in SEQ ID NO: 7; and a VL comprising an amino acid sequence which is at least 90%, 95%, 96%, 97%, 98%, 99%, or 100% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a heavy chain constant region selected from the group consisting of human IgG1, IgG2, IgG3, IgG4, IgA1, and IgA2. In an embodiment, the heavy chain constant region is IgG1. In an embodiment, the heavy chain constant region is IgG2. In an embodiment, the antibody comprises a light chain constant region selected from the group consisting of a human kappa light chain constant region and a human lambda light chain constant region.

In an embodiment, the antibody comprises an IgG₁ heavy chain constant region. In an embodiment, the amino acid sequence of the IgG₁ heavy chain constant region comprises S239D/I332E mutations, numbered according to the EU numbering system. In an embodiment, the amino acid sequence of the IgG₁ heavy chain constant region comprises S239D/A330L/I332E mutations, numbered according to the EU numbering system. In an embodiment, the amino acid sequence of the IgG₁ heavy chain constant region comprises L235V/F243L/R292P/Y300L/P396L mutations, numbered according to the EU numbering system. In an embodiment, the IgG₁ heavy chain constant region is afucosylated IgG₁.

In an embodiment, the antibody comprises a heavy chain comprising the amino acid sequence set forth in SEQ ID NO: 9. In an embodiment, the antibody comprises a heavy chain comprising the amino acid sequence set forth in SEQ ID NO: 9 and a light chain comprising an amino acid sequence which is at least 90%, 95%, 96%, 97%, 98%, 99%, or 100% identical to the amino acid sequence set forth in SEQ ID NO: 10.

In an embodiment, the antibody comprises a heavy chain comprising the amino acid sequence set forth in SEQ ID NO: 9 and a light chain comprising the amino acid sequence set forth in SEQ ID NO: 10. In an embodiment, the amino acid sequence of the heavy chain consists of the amino acid sequence set forth in SEQ ID NO: 9 and the amino acid sequence of the light chain consists of the amino acid sequence set forth in SEQ ID NO: 10.

In an embodiment the antibody is botensilimab (a.k.a. AGEN1181), the amino acid sequences of which are provided in Table 2 below.

TABLE 2
Amino acid sequences of botensilimab

| Description | SEQ ID NO: | Amino acid sequence |
|---|---|---|
| CDRH1 | 1 | SYSMN |
| CDRH2 | 2 | SISSSSSYIYYAESVKG |
| CDRH3 | 3 | VGLFGPFDI |
| CDRL1 | 4 | RASQSVSRYLG |
| CDRL2 | 5 | GASTRAT |
| CDRL3 | 6 | QQYGSSPWT |
| VH | 7 | EVQLVESGGGLVKPGGSLRLSCAASGFTFSSYSMNWVRQAPG |
| | | KGLEWVSSISSSSSYIYYAESVKGRFTISRDNAKNSLYLQMNSL |
| | | RAEDTAVYYCARVGLFGPFDIWGQGTLVTVSS |
| VL | 8 | EIVLTQSPGTLSLSPGERATLSCRASQSVSRYLGWYQQKPGQAP |
| | | RLLIYGASTRATGIPDRFSGSGSGTDFTLTITRLEPEDFAVYYCQ |
| | | QYGSSPWTFGQGTKVEIK |
| Heavy chain | 9 | EVQLVESGGGLVKPGGSLRLSCAASGFTFSSYSMNWVRQAPG |
| | | KGLEWVSSISSSSSYIYYAESVKGRFTISRDNAKNSLYLQMNSL |
| | | RAEDTAVYYCARVGLFGPFDIWGQGTLVTVSSASTKGPSVFPL |
| | | APSSKSTSGGTAALGCLVKDYFPEPVTVSWNSGALTSGVHTFP |
| | | AVLQSSGLYSLSSVVTVPSSSLGTQTYICNVNHKPSNTKVDKR |
| | | VEPKSCDKTHTCPPCPAPELLGGPDVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPLPEEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPG |
| Light chain | 10 | EIVLTQSPGTLSLSPGERATLSCRASQSVSRYLGWYQQKPGQAP |
| | | RLLIYGASTRATGIPDRFSGSGSGTDFTLTITRLEPEDFAVYYCQ |
| | | QYGSSPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |

### Anti-PD-1 Antibodies

In an embodiment, any one of the methods disclosed herein further comprises administering an antibody that specifically binds to human PD-1 to the subject. Antibodies that specifically bind to human PD-1 (i.e., anti-PD-1 antibodies) that are useful in the methods and uses described herein include but are not limited to those listed below.

In an embodiment, the antibody that specifically binds to human PD-1 comprises: a heavy chain variable region (VH) comprising the CDRH1, CDRH2, and CDRH3 amino acid sequences of the VH amino acid sequence set forth in SEQ ID NO: 17. In an embodiment, the antibody that specifically binds to human PD-1 comprises: a light chain variable region (VL) comprising the CDRL1, CDRL2, and CDRL3 amino acid sequences of the VL amino acid sequence set forth in SEQ ID NO: 18. In an embodiment, the antibody that specifically binds to human PD-1 comprises a heavy chain variable region (VH) comprising the CDRH1, CDRH2, and CDRH3 amino acid sequences of the VH amino acid sequence set forth in SEQ ID NO: 17 and a light chain variable region (VL) comprising the CDRL1, CDRL2, and CDRL3 amino acid sequences of the VL amino acid sequence set forth in SEQ ID NO: 18.

In an embodiment, the antibody that specifically binds to human PD-1 comprises the CDRH1, CDRH2, and CDRH3 amino acid sequences set forth in SEQ ID NO: 11, 12, and 13, respectively. In an embodiment, the antibody that specifically binds to human PD-1 comprises the CDRL1, CDRL2, and CDRL3 amino acid sequences set forth in SEQ ID NO: 14, 15, and 16, respectively. In an embodiment, the antibody that specifically binds to human PD-1 comprises the CDRH1, CDRH2, CDRH3, CDRL1, CDRL2, and CDRL3 amino acid sequences set forth in SEQ ID NOs: 11, 12, 13, 14, 15, and 16, respectively.

In an embodiment, the antibody that specifically binds to human PD-1 comprises a VH comprising the amino acid sequence set forth in SEQ ID NO: 17. In an embodiment, the antibody that specifically binds to human PD-1 comprises a VL comprising the amino acid sequence set forth in SEQ ID NO: 18. In an embodiment, the antibody that specifically binds to human PD-1 comprises: a VH comprising the amino acid sequence set forth in SEQ ID NO: 17; and a VL comprising an amino acid sequence which is at least 90%, 95%, 96%, 97%, 98%, 99%, or 100% identical to the amino acid sequence set forth in SEQ ID NO: 18.

In an embodiment, the antibody that specifically binds to human PD-1 comprises a VH comprising the amino acid sequence set forth in SEQ ID NO: 17 and a VL comprising the amino acid sequence set forth in SEQ ID NO: 18.

In an embodiment, the antibody that specifically binds to human PD-1 comprises a heavy chain constant region selected from the group consisting of human IgG1, IgG2, IgG3, IgG4, IgA1, and IgA2. In an embodiment, the heavy chain constant region is IgG1. In an embodiment, the heavy chain constant region is IgG2. In an embodiment, the antibody comprises a light chain constant region selected from the group consisting of a human kappa light chain constant region and a human lambda light chain constant region.

In an embodiment, the antibody comprises a heavy chain comprising the amino acid sequence set forth in SEQ ID NO: 19. In an embodiment, the antibody comprises a light chain comprising the amino acid sequence set forth in SEQ ID NO: 20. In an embodiment, the antibody comprises a heavy chain comprising the amino acid sequence set forth in SEQ ID NO: 19 and a light chain comprising an amino acid sequence which is at least 90%, 95%, 96%, 97%, 98%, 99%, or 100% identical to the amino acid sequence set forth in SEQ ID NO: 20.

In an embodiment, the antibody that specifically binds to human PD-1 comprises a heavy chain comprising the amino acid sequence set forth in SEQ ID NO: 19 and a light chain comprising the amino acid sequence set forth in SEQ ID NO: 20. In an embodiment, the amino acid sequence of the heavy chain consists of the amino acid sequence set forth in SEQ ID NO: 19 and the amino acid sequence of the light chain consists of the amino acid sequence set forth in SEQ ID NO: 20.

In an embodiment the antibody that specifically binds to human PD-1 is balstilimab (a.k.a. AGEN2034), the amino acid sequences of which are provided in Table 3 below.

TABLE 3
Amino acid sequences of balstilimab

| Description | SEQ ID NO: | Amino acid sequence |
|---|---|---|
| CDRH1 | 11 | SYGMH |
| CDRH2 | 12 | VIWYDGSNKYYADSVKG |
| CDRH3 | 13 | NGDH |
| CDRL1 | 14 | RASQSVSSNLA |
| CDRL2 | 15 | GASTRAT |
| CDRL3 | 16 | QQYNNWPRT |
| VH | 17 | QVQLVESGGGVVQPGRSLRLSCAASGFTFSSYGMHWVRQAPG |
| | | KGLEWVAVIWYDGSNKYYADSVKGRFTISRDNSKNTLYLQM |
| | | NSLRAEDTAVYYCASNGDHWGQGTLVTVSS |
| VL | 18 | EIVMTQSPATLSVSPGERATLSCRASQSVSSNLAWYQQKPGQA |
| | | PRLLIYGASTRATGIPARFSGSGSGTEFTLTISSLQSEDFAVYYC |
| | | QQYNNWPRTFGQGTKVEIK |
| Heavy chain | 19 | QVQLVESGGGVVQPGRSLRLSCAASGFTFSSYGMHWVRQAPG |
| | | KGLEWVAVIWYDGSNKYYADSVKGRFTISRDNSKNTLYLQM |
| | | NSLRAEDTAVYYCASNGDHWGQGTLVTVSSASTKGPSVFPLA |
| | | PCSRSTSESTAALGCLVKDYFPEPVTVSWNSGALTSGVHTFPA |
| | | VLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVV |
| | | VDVSQEDPEVQFNWYVDGVEVHNAKTKPREEQFNSTYRVVS |
| | | VLTVLHQDWLNGKEYKCKVSNKGLPSSIEKTISKAKGQPREPQ |
| | | VYTLPPSQEEMTKNQVSLTCLVKGFYPSDIAVEWESNGQPENN |
| | | YKTTPPVLDSDGSFFLYSRLTVDKSRWQEGNVFSCSVMHEALH |
| | | NHYTQKSLSLSLG |
| Light chain | 20 | EIVMTQSPATLSVSPGERATLSCRASQSVSSNLAWYQQKPGQA |
| | | PRLLIYGASTRATGIPARFSGSGSGTEFTLTISSLQSEDFAVYYC |
| | | QQYNNWPRTFGQGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |

### Methods of Treatment

The instant disclosure demonstrates that antibodies that specifically bind to human CTLA-4 (e.g., botensilimab) are highly effective in treating colorectal cancer. The instant disclosure also demonstrates that antibodies that specifically bind to human CTLA-4 (e.g., botensilimab) are highly effective in treating metastatic, unresectable colorectal adenocarcinoma (e.g., non-MSI-H/dMMR metastatic colorectal adenocarcinoma). Accordingly, the instant disclosure is broadly directed to methods for treating colorectal cancer with an antibody that specifically binds to human CTLA-4. Also provided herein are particular dosage regimens for administering an antibody that specifically binds to human CTLA-4 that results in a reduction of tumor burden in the subject.

In an aspect, provided herein is a method of treating colorectal cancer in a subject in need thereof, the method comprising administering to the subject an effective amount of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has colorectal cancer, the method comprising administering to the subject an effective amount of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating colorectal cancer in a subject in need thereof, the method comprising administering to the subject an antibody that specifically binds to human Cytotoxic T-Lymphocyte Antigen 4 (CTLA-4) at a dose of about 25 mg to about 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has colorectal cancer, the method comprising administering to the subject an antibody that specifically binds to human CTLA-4 at a dose of about 25 mg to about 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody is administered at a dose of about 50 mg to about 175 mg. In an embodiment, the antibody is administered at a dose of about 25 mg to about 150 mg. In an embodiment, the antibody is administered at a dose of about 50 mg to about 150 mg. In an embodiment, the antibody is administered at a dose of about 75 mg to about 150 mg.

In an embodiment, the antibody is administered at a dose of 50 mg to 175 mg. In an embodiment, the antibody is administered at a dose of 25 mg to 150 mg. In an embodiment, the antibody is administered at a dose of 50 mg to 150 mg. In an embodiment, the antibody is administered at a dose of 75 mg to 150 mg.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg.

In an embodiment, the antibody is administered intravenously. In an embodiment, the antibody is administered intratumorally. In an embodiment, the antibody is administered peritumorally.

In an embodiment, the antibody is administered by intravenous infusion over about 30 minutes. In an embodiment, the antibody is administered by intravenous infusion over about 45 minutes. In an embodiment, the antibody is administered by intravenous infusion over about 60 minutes. In an embodiment, the antibody is administered by intravenous infusion over about 90 minutes.

In an embodiment, the antibody is administered about once weekly. In an embodiment, the antibody is administered about once every 2 weeks. In an embodiment, the antibody is administered about once every 3 weeks. In an embodiment, the antibody is administered about once every 4 weeks. In an embodiment, the antibody is administered about once every 5 weeks. In an embodiment, the antibody is administered about once every 6 weeks. In an embodiment, the antibody is administered about once every 7 weeks. In an embodiment, the antibody is administered about once every 8 weeks. In an embodiment, the antibody is administered about once every 9 weeks. In an embodiment, the antibody is administered about once every 10 weeks. In an embodiment, the antibody is administered about once every 11 weeks. In an embodiment, the antibody is administered about once every 12 weeks. In an embodiment, the antibody is administered about once every 13 weeks.

In an embodiment, the antibody is administered once weekly. In an embodiment, the antibody is administered once every 2 weeks. In an embodiment, the antibody is administered once every 3 weeks. In an embodiment, the antibody is administered once every 4 weeks. In an embodiment, the antibody is administered once every 5 weeks. In an embodiment, the antibody is administered once every 6 weeks. In an embodiment, the antibody is administered once every 7 weeks. In an embodiment, the antibody is administered once every 8 weeks. In an embodiment, the antibody is administered once every 9 weeks. In an embodiment, the antibody is administered once every 10 weeks. In an embodiment, the antibody is administered once every 11 weeks. In an embodiment, the antibody is administered once every 12 weeks. In an embodiment, the antibody is administered once every 13 weeks.

In an embodiment, the antibody is administered intravenously at a dose of about 25 mg once every 6 weeks. In an embodiment, the antibody is administered intravenously at a dose of about 50 mg once every 6 weeks. In an embodiment, the antibody is administered intravenously at a dose of 75 mg once every 6 weeks. In an embodiment, the antibody is administered intravenously at a dose of 100 mg once every 6 weeks. In an embodiment, the antibody is administered intravenously at a dose of 150 mg once every 6 weeks.

In an embodiment, the dose is a therapeutically effective amount.

In an embodiment, the colorectal cancer is colorectal adenocarcinoma. In an embodiment, the colorectal cancer is metastatic. In an embodiment, the colorectal cancer is unresectable. In an embodiment, the colorectal cancer is metastatic, unresectable colorectal adenocarcinoma. In an embodiment, the subject does not have liver metastases. In an embodiment, the subject has liver metastases.

In an embodiment, the antibody is administered to the subject prior to surgical resection of a primary tumor. In an embodiment, the subject has not received any prior chemotherapy. In an embodiment, the subject has not received any prior radiation therapy.

In an embodiment, the colorectal cancer is relapsed and/or refractory. In an embodiment, the subject has received at least one prior chemotherapy. In an embodiment, the at least one prior chemotherapy is fluoropyrimidine, irinotecan, oxaliplatin, or an anti-EGFR antibody. In an embodiment, the anti-EGFR antibody is cetuximab or panitumumab.

In an embodiment, the subject has previously been treated with folinic acid, 5-fluorouracil, oxaliplatin, and irinotecan (i.e., FOLFOXIRI therapy).

In an embodiment, the subject is unable to tolerate a standard of care treatment.

In an embodiment, the subject has a RAS mutation. In an embodiment, the RAS mutation is a KRAS or NRAS mutation.

In an embodiment, the colorectal cancer is not microsatellite instable—high (MSI-H). In an embodiment, the colorectal cancer is microsatellite stable (MSS). In an embodiment, the colorectal cancer is not mismatch repair deficient (dMMR).

In an embodiment, the subject has not received prior immune checkpoint inhibitor therapy. In an embodiment, the subject has not previously been treated with an anti-PD-1, anti-PD-L1, or anti-CTLA-4 antibody.

In an embodiment, the subject has received prior immune checkpoint inhibitor therapy. In an embodiment, the subject has previously been treated with an anti-PD-1, anti-PD-L1, or anti-CTLA-4 antibody.

In an embodiment, the cancer may be positive or negative for expression of PD-L1 (e.g., when PD-L1 stained tumor infiltrating immune cells cover less than 5% of the tumor area as determined by immunohistochemistry or as determined by any commercially available companion diagnostic, including diagnostics with FDA premarket approval numbers P160002, P150013, or P150025).

In an embodiment, the subject has not received prior regorafenib, trifluridine, and/or tipiracil therapy.

In an embodiment, the cancer is refractory to a standard of care treatment. In an embodiment, the standard of care treatment is chemotherapy or radiation. In an embodiment, the standard of care treatment is folinic acid, 5-fluorouracil, oxaliplatin, irinotecan, fluoropyrimidine, cetuximab, and/or panitumumab.

In an embodiment, the administration of the antibody reduces tumor size in the subject. In an embodiment, the administration of the antibody increases T-cell, memory T cell, myeloid cell, and/or antigen presenting cell activation in the subject. In an embodiment, the administration of the antibody reduces the number of Treg cells in the subject. In an embodiment, the administration of the antibody increases expansion of new T cell clones.

In an embodiment, the administration of the antibody increases the level of one or more cytokine. In an embodiment, the one or more cytokine is CXCL9 or CXCL10. In an embodiment, the administration of the antibody increases the level of IFN-γ, HLA-DR, and/or ICOS expression.

In an embodiment, before administration of the antibody the subject has measurable disease on baseline imaging per RECIST 1.1.

In an embodiment, before administration of the antibody the subject has an Eastern Cooperative Oncology Group performance status (PS) 0-1. In an embodiment, before administration of the antibody the subject has a predicted life expectancy of ≥12 weeks. In an embodiment, before administration of the antibody the subject has: adequate organ function as defined by one or more of: a) neutrophils≥1500/μL; b) platelets≥100×103/μL; c) hemoglobin ≥8.0 g/dL; d) creatinine clearance≥30 mL/min as measured or calculated per local institutional standards; e) AST/ALT≤2.5×upper limit of normal (ULN); f) total bilirubin≤1.5×ULN (except patients with Gilbert syndrome who must have a total bilirubin level of ≤3.0×ULN); and/or g) albumin≥3.0 g/dL.

In an embodiment, the subject does not have partial or complete bowel obstruction within the last 3 months, signs/symptoms of bowel obstruction, or known radiologic evidence of impending obstruction.

In an embodiment, the subject does not have refractory ascites defined as requiring 2 or more therapeutic paracenteses within the last 4 weeks or ≥4 times within the last 90 days or ≥1 time within the last 2 weeks prior to administration of the antibody.

In an embodiment, the subject does not have clinically significant cardiovascular disease. In an embodiment, the subject does not have active brain metastases or leptomeningeal metastases.

In an embodiment, the subject does not have a concurrent malignancy that requires treatment or a history of prior malignancy that was active within 2 years prior to administration of the antibody.

In an embodiment, the subject has not had cytotoxic therapy, targeted therapy, or other investigational therapy within 3 weeks prior to administration of the antibody.

In an embodiment, the subject has not had other monoclonal antibody, antibody-drug conjugate, or radioimmunoconjugate therapy within 4 weeks prior to administration of the antibody.

In an embodiment, the subject has not had small molecule tyrosine kinase inhibitor therapy within 2 weeks prior to administration of the antibody.

In an embodiment, the objective response rate (ORR), duration of response (DOR), disease control rate (DCR), and progression-free survival (PFS) are assessed for a subject according to the Response Evaluation Criteria in Solid Tumors Version 1.1 (RECIST 1.1).

In an embodiment, the method results in a complete response, as defined by RECIST 1.1. In an embodiment, the method results in a partial response, as defined by RECIST 1.1. In an embodiment, the method results in a stable disease, as defined by RECIST 1.1.

In an embodiment, the method results in about a 1, 5, 10, 20, 30, 40, 50, 60, 70, 80, 90, or 100% reduction in tumor burden in the subject. In an embodiment, the method results in no change in tumor burden in the subject. In an embodiment, the method results in about a 1% reduction in tumor burden in the subject. In an embodiment, the method results in about a 5% reduction in tumor burden in the subject. In an embodiment, the method results in about a 10% reduction in tumor burden in the subject. In an embodiment, the method results in about a 20% reduction in tumor burden in the subject. In an embodiment, the method results in about a 30% reduction in tumor burden in the subject. In an embodiment, the method results in about a 40% reduction in tumor burden in the subject. In an embodiment, the method results in about a 50% reduction in tumor burden in the subject. In an embodiment, the method results in about a 60% reduction in tumor burden in the subject. In an embodiment, the method results in about a 70% reduction in tumor burden in the subject. In an embodiment, the method results in about an 80% reduction in tumor burden in the subject. In an embodiment, the method results in about a 90% reduction in tumor burden in the subject. In an embodiment, the method results in about a 100% reduction in tumor burden in the subject.

In an embodiment, the method results in a reduced tumor burden. In an embodiment, the method results in increased survival. In an embodiment, the method results in an increase in overall survival. In an embodiment, the method results in an increase in progression-free survival.

In an aspect, provided herein is a method of treating metastatic colorectal adenocarcinoma in a subject in need thereof, the method comprising administering to the subject an antibody that specifically binds to human CTLA-4 at a dose of 75 mg or 150 mg once every 6 weeks, wherein the antibody comprises a VH comprising the amino acid sequence set forth in SEQ ID NO: 7 and a VL comprising the amino acid sequence set forth in SEQ ID NO: 8.

In an aspect, provided herein is a method of treating metastatic colorectal adenocarcinoma in a subject in need thereof, the method comprising administering to the subject: a) an antibody that specifically binds to human CTLA-4 at a dose of 75 mg once every 6 weeks, wherein the antibody comprises a VH comprising the amino acid sequence set forth in SEQ ID NO: 7 and a VL comprising the amino acid sequence set forth in SEQ ID NO: 8; and b) an antibody that specifically binds to human PD-1 at a dose of 240 mg once every 2 weeks, wherein the antibody comprises a VH comprising the amino acid sequence set forth in SEQ ID NO: 17 and a VL comprising the amino acid sequence set forth in SEQ ID NO: 18.

In an aspect, provided herein is a method of treating metastatic colorectal adenocarcinoma in a subject in need thereof, the method comprising administering to the subject: a) an antibody that specifically binds to human CTLA-4 at a dose of 150 mg once every 6 weeks, wherein the antibody comprises a VH comprising the amino acid sequence set forth in SEQ ID NO: 7 and a VL comprising the amino acid sequence set forth in SEQ ID NO: 8; and b) an antibody that specifically binds to human PD-1 at a dose of 240 mg once every 2 weeks, wherein the antibody comprises a VH comprising the amino acid sequence set forth in SEQ ID NO: 17 and a VL comprising the amino acid sequence set forth in SEQ ID NO: 18.

In an aspect, provided herein is a method of treating metastatic colorectal adenocarcinoma in a subject in need thereof, the method comprising administering to the subject: a) an antibody that specifically binds to human CTLA-4 at a dose of 75 mg once every 6 weeks, wherein the antibody comprises a VH comprising the amino acid sequence set forth in SEQ ID NO: 7 and a VL comprising the amino acid sequence set forth in SEQ ID NO: 8; and b) an antibody that specifically binds to human PD-1 at a dose of 450 mg once every 3 weeks, wherein the antibody comprises a VH comprising the amino acid sequence set forth in SEQ ID NO: 17 and a VL comprising the amino acid sequence set forth in SEQ ID NO: 18.

In an aspect, provided herein is a method of treating metastatic colorectal adenocarcinoma in a subject in need thereof, the method comprising administering to the subject: a) an antibody that specifically binds to human CTLA-4 at a dose of 150 mg once every 6 weeks, wherein the antibody comprises a VH comprising the amino acid sequence set forth in SEQ ID NO: 7 and a VL comprising the amino acid sequence set forth in SEQ ID NO: 8; and b) an antibody that specifically binds to human PD-1 at a dose of 450 mg once every 3 weeks, wherein the antibody comprises a VH comprising the amino acid sequence set forth in SEQ ID NO: 17 and a VL comprising the amino acid sequence set forth in SEQ ID NO: 18.

In an embodiment, the metastatic colorectal adenocarcinoma is unresectable. In an embodiment, the metastatic colorectal adenocarcinoma is non-MSI-H/dMMR.

In an embodiment, any one of the methods disclosed herein further comprises administering an antibody that specifically binds human PD-1 to the subject. In an embodiment, the antibody that specifically binds to human PD-1 is administered at a dose of 200 mg to 500 mg. In an embodiment, the antibody that specifically binds to human PD-1 is administered at a dose of 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg. In an embodiment, the antibody that specifically binds to human PD-1 is administered at a dose of 240 mg. In an embodiment, the antibody that specifically binds to human PD-1 is administered at a dose of 450 mg.

In an embodiment, the antibody that specifically binds to human PD-1 is administered once weekly, once every 2 weeks, or once every 3 weeks. In an embodiment, the antibody that specifically binds to human PD-1 is administered at a dose of 240 mg once every 2 weeks. In an embodiment, the antibody that specifically binds to human PD-1 is administered at a dose of 450 mg once every 3 weeks.

In an embodiment, any one of the methods disclosed herein further comprises administering an antibody that specifically binds human PD-1 at a dose of about 240 mg once every 2 weeks. In an embodiment, any one of the methods disclosed herein further comprises administering an antibody that specifically binds human PD-1 at a dose of 240 mg once every 2 weeks. In an embodiment, any one of the methods disclosed herein further comprises administering an antibody that specifically binds human PD-1 at a dose of about 450 mg once every 3 weeks. In an embodiment, any one of the methods disclosed herein further comprises administering an antibody that specifically binds human PD-1 at a dose of 450 mg once every 3 weeks.

In an embodiment, any one of the methods disclosed herein further comprises administering balstilimab to the subject. In an embodiment, any one of the methods disclosed herein further comprises administering balstilimab at a dose of about 240 mg once every 2 weeks to the subject. In an embodiment, any one of the methods disclosed herein further comprises administering balstilimab at a dose of 240 mg once every 2 weeks to the subject. In an embodiment, any one of the methods disclosed herein further comprises administering balstilimab at a dose of about 450 mg once every 3 weeks to the subject. In an embodiment, any one of the methods disclosed herein further comprises administering balstilimab at a dose of 450 mg once every 3 weeks to the subject.

In an embodiment, the antibody that specifically binds human PD-1 is administered intravenously. In an embodiment, the antibody that specifically binds human PD-1 is administered by intravenous infusion over about 30 minutes.

In an embodiment, the antibody that specifically binds human PD-1 is administered prior to the antibody that specifically binds human CTLA-4.

In an aspect, provided herein is an antibody that specifically binds to human CTLA-4 for use in the treatment of colorectal cancer, wherein the treatment is performed according to a method disclosed herein.

In an aspect, provided herein is an antibody that specifically binds to human CTLA-4 for use in the manufacture of a medicament for the treatment of colorectal cancer, wherein the treatment is performed according to a method disclosed herein.

Use of an antibody that specifically binds to human CTLA-4 for the treatment of colorectal cancer, wherein the treatment is performed according to the method of any one of the previous claims.

In an aspect, provided herein is an antibody that specifically binds to human CTLA-4 and an antibody that specifically binds to human PD-1 for use in the treatment of colorectal cancer, wherein the treatment is performed according to the method of any one of the previous claims.

In an aspect, provided herein is an antibody that specifically binds to human CTLA-4 and an antibody that specifically binds to human PD-1 for use in the manufacture of a medicament for the treatment of colorectal cancer, wherein the treatment is performed according to the method of any one of the previous claims.

In an aspect, provided herein is a use of an antibody that specifically binds to human CTLA-4 and an antibody that specifically binds to human PD-1 for the treatment of colorectal cancer, wherein the treatment is performed according to the method of any one of the previous claims.

### Examples

### Example 1—Phase 2 Study of Botensilimab (AGEN1181) as Monotherapy and in Combination with Balstilimab (AGEN2034) for the Treatment of Refractory Metastatic Colorectal Cancer

This Phase 2 study further evaluated the safety and efficacy of botensilimab as monotherapy and in combination with balstilimab (AGEN2034, an anti-PD-1 antibody) in patients with adenocarcinoma of the colon or rectum who had received at least 1 prior chemotherapy for metastatic or recurrent disease. The study also aimed to optimize the dose of botensilimab in patients with metastatic CRC.

a. Study Design

### Overall Design

This study was a multicenter, randomized, open-label, Phase 2 study of botensilimab as monotherapy and in combination with balstilimab, or Investigator's choice standard of care (SoC), regorafenib or trifluridine and tipiracil. The study enrolled patients with adenocarcinoma of the colon or rectum who had received at least 1 prior chemotherapy for metastatic or recurrent disease.

The patients included in the study were those who had progressed on or who were intolerant to fluoropyrimidine, irinotecan, oxaliplatin, and if applicable, anti-EGFR directed therapy. Most patients had received more than one prior line of therapy. Only RAS mutant patients who received FOLFOXIRI in the first line were eligible for this study as early as the second line. The study excluded patients with active liver metastases.

Eligible patients were randomized 1:1:1:1:1 to Arm A, Arm B, Arm C, Arm D, or Arm E. Baseline study procedures included radiographic imaging studies (computed tomography [CT] or magnetic resonance imaging [MRI] of the chest, abdomen, and pelvis [C/A/P], central nervous system [CNS] imaging for patients with a history of brain metastases) and routine laboratory testing.

### Study Treatments

### Treatment Arms Included:

Arm A: Botensilimab 75 mg IV Q6W for up to 4 doses and balstilimab 240 mg IV Q2W. Balstilimab could be continued for up to 2 years (17 six-week cycles).
Arm B: Botensilimab 150 mg IV Q6W for up to 4 doses and balstilimab 240 mg IV Q2W. Balstilimab could be continued for up to 2 years (17 six-week cycles).
Arm C: Botensilimab 75 mg IV Q6W for up to 4 doses.
Arm D: Botensilimab 150 mg IV Q6W for up to 4 doses.
Arm E: Investigator choice of SoC:
   Regorafenib orally:
      160 mg (4×40 mg tablets), once daily for the first 21 days of each 28-day cycle, OR
      80 mg (2×40 mg tablets) once daily on Days 1 to 7 in Cycle 1, followed by 120 mg (3×40 mg tablets) once daily on Days 8 to 14 in Cycle 1, followed by 160 mg (4×40 mg tablets) once daily on Days 15 to 21 in Cycle 1 (28-day cycle). Subsequent 28-day cycles consisted of 160 mg (4×40 mg tablets) once daily on Days 1 to 21.
   35 mg/m²/dose trifluridine and tipiracil [rounded to the 5 mg increment] orally twice daily on Days 1 through 5 and Days 8 through 12 of each 28-day cycle.

TABLE 4
Study Treatments

| | Study Treatment Name: | | | |
|---|---|---|---|---|
| | | | | Trifluridine and |
| | Botensilimab | Balstilimab | Regorafenib | Tipiracil |
| Dosage | Solution for | Solution for | Tablet | Tablet |
| Formulation: | infusion | infusion | | |
| Unit Dosage | 50 mg/mL | 50 mg/mL | 40 mg | 15 mg trifluridine/ |
| Strength: | | | | 6.14 mg tipiracil |
| | | | | 20 mg trifluridine/ |
| | | | | 8.19 mg tipiracil |
| Dosage Level: | 75 mg or | 240 mg | 80 mg (2 × 40 mg tablets) | 35 mg/m²/dose |
| | 150 mg | | 120 mg (3 × 40 mg tablets) | trifluridine and |
| | | | or 160 mg | tipiracil (rounded to |
| | | | (4 × 40 mg tablets) | the 5 mg increment) |
| Route of | intravenous | intravenous | Oral | Oral |
| Administration: | infusion | infusion | | |

During the treatment period, patients had routine clinical visits for administration of study treatment and monitoring of safety, well-being, and changes in disease status.

Anti-tumor efficacy was determined via imaging assessments, which were performed at 8 weeks, 16 weeks, 24 weeks, and every 12 weeks thereafter. Disease response evaluation was performed per the investigator, and may have been evaluated by independent central review per the Sponsor discretion.

Safety was assessed via monitoring of AEs, SAEs, treatment discontinuation due to AEs, physical examinations, vital signs, hematology, and chemistry laboratories. Patients who discontinued study treatment for reasons other than progressive disease (PD) could continue with imaging assessments.

For Survival Follow-up, patients who were no longer continuing study visits and imaging were followed every 12 weeks for survival status via telephone call. Futility analyses were conducted on both botensilimab arms at prespecified timepoints, with the provision that if one arm was closed, the other arm could continue.

In the monotherapy arms, patients were treated for approximately 24 weeks or until one of the criteria for discontinuation of study treatment was met. In the combination treatment arms, patients were treated with balstilimab for up to 2 years (17 six-week cycles), or until one of the criteria for discontinuation of study treatment was met. In the SoC arm, patients were treated with regorafenib or trifluridine and tipiracil until disease progression or unacceptable toxicity.

### Study Drug Administration

Botensilimab was administered via IV infusion over 30 (±5) minutes as a monotherapy. Patients were observed for 30 minutes after the end of the infusion. Measurement of vital signs was performed at each cycle prior to starting each infusion and at the end of each infusion. Infusions were followed immediately with a saline flush of the IV line, per institutional guidelines.

In the combination arm, balstilimab was administered prior to botensilimab. Balstilimab was administered via IV infusion over 30 (±5) minutes. Patients were observed for 30 minutes post-balstilimab infusion for IRRs (botensilimab was not administered during this observation period). Botensilimab was administered via IV infusion over 30 (±5) minutes. Patients were observed for 30 minutes after the end of the infusion. Measurement of vital signs was performed at each cycle prior to starting each infusion and at the end of each infusion. Infusions were followed immediately with a saline flush of the IV line, per institutional guidelines.

If a dose level of botensilimab (monotherapy or combination) was modified by the SMC and a subsequent different dose level was opened, patients had the option per Investigator decision and Sponsor approval to dose modify botensilimab to the new dose level. Dose reductions and/or escalations were not allowed on this study for botensilimab or balstilimab for any other reasons; patients received the full dose of botensilimab and balstilimab as per protocol or until treatment was held or discontinued as appropriate. If treatment was held and the patient was ready to resume treatment, the subsequent treatment occurred on the next protocol specified treatment date, i.e., treatments were skipped, not delayed and given off-schedule.

### Number Of Patients

Approximately 230 patients were enrolled: approximately 30 patients per arm, allowing for expansion up to approximately 50 patients per botensilimab arms. Any patients discontinuing from the study were not replaced. Existing arms could be stopped early for futility, and additional patients could be enrolled at different dose levels of botensilimab per SMC recommendations.

### Treatment Beyond Disease Progression

Patients who were tolerating the drug and were considered by the Investigator to be deriving clinical benefit were permitted, with the Sponsor's approval, to continue with treatment beyond initial RECIST 1.1-defined PD if they met the following clinical stability criteria:
Absence of clinical symptoms and signs (including worsening of laboratory values) indicating PD, i.e., patient was clinically stable per Investigator judgment.
No decline in Eastern Cooperative Oncology Group (ECOG) performance status attributed to underlying malignancy.
Absence of rapid progression of disease or progressive tumor at critical anatomical sites (e.g., cord compression) requiring urgent alternative medical intervention.

Patients treated beyond progression had to first provide written informed consent using a designated ICF prior to receiving additional study drug infusions. New lesions were considered measurable at the time of initial progression if the longest diameter was ≥10 mm (except for pathological lymph nodes, which must have had a short axis of ≥15 mm). Any new lesion considered non-measurable at the time of initial progression could become measurable and therefore be included in the tumor burden measurement if the longest diameter increased to ≥10 mm (except for pathological lymph nodes, which must have had an increase in short axis to ≥15 mm).

### Treatment Duration

In Arms A to D, patients received up to 4 doses of study drug over 24 weeks in the monotherapy arms and study treatment for up to 2 years (17 six-week cycles) in the combination arms, until any disease progression (with exceptions noted as above), unacceptable toxicity, or patient wished to withdraw consent for any reason. In Arm E (i.e., SoC), patients received study drug until disease progression or unacceptable toxicity.

After treatment completion or discontinuation (e.g., due to toxicity), patients were followed for safety at 30 days and 90 days, and for long-term follow-up every 3 months until the end of the study or the patient discontinued the study.

### Prohibited Medications and Treatments

Medications or vaccinations specifically prohibited in the exclusion criteria were not allowed during the ongoing study. If there was a clinical indication for any medication or vaccination specifically prohibited during the study, discontinuation from study therapy or vaccination could have been required. The final decision on any supportive therapy or vaccination rested with the Investigator and/or the patient's primary physician. However, the decision to continue the patient on study treatment required the mutual agreement of the Investigator, the Sponsor, and the patient.

Listed below are specific restrictions for concomitant therapy during the course of the study:
Antineoplastic systemic chemotherapy or biological therapy other than SoC.
Immuno-oncology therapies not specified in this protocol.
Investigational agents other than the assigned study treatment.
Live vaccines.
Systemic glucocorticoids (>10 mg prednisone equivalent for >1 week) for any purpose other than to treat an immune-related adverse event. Note: Use of prophylactic corticosteroids to avoid allergic reactions (e.g., IV contrast dye or transfusions) was permitted, as was the use of inhaled steroids or intranasal or local injection of corticosteroids.

In addition to the above, patients taking regorafenib had to avoid strong CYP3A4 inducers or inhibitors.

Patients treated with any prohibited medication (excluding the exceptions noted above) for clinical management were removed from the study. Rare exceptions were considered regarding the use of glucocorticoids for reasons other than irAE as approved by the Medical Monitor. All treatments that the Investigator considered necessary for a patient's welfare could be administered at the discretion of the Investigator in keeping with the community standards of medical care. All concomitant medications were recorded on the electronic case report form (eCRF) including all prescription, over-the-counter products, herbal supplements, and IV medications and fluids. If changes occurred during the study period, documentation of drug dosage, frequency, route, and date were noted in the eCRF.

### Surgery and Radiation Therapy

If a patient required surgery for management of progressive malignant disease, they were discontinued from study treatment. In the instance of surgery for bowel obstruction, if there was not confirmed PD, the patient could continue receiving treatment on study. Palliative radiotherapy on non-target lesions was permitted following discussion with the Sponsor based on the specific clinical scenario.

### Permanent Discontinuation Of Study Drug Treatment

Botensilimab or balstilimab treatment was permanently discontinued for any of the following reasons:
Occurrence of an immune-related adverse event that meets the criteria for discontinuation.
Confirmed PD unless the patient was considered by the Investigator to derive clinical benefit from the treatment, the patient was clinically stable, and there was approval from the Sponsor.
Clinical progression, in absence of radiologic progression on the basis of RECIST 1.1 as suggested by one or more of the following:
   Signs and/or symptoms consistent with clinically significant progression of disease, including worsening of laboratory values, appearance of new lesion(s)/worsening of the lesion best seen clinically, etc.
   Decline in ECOG performance status due to worsening malignancy.
   Tumor progression at critical anatomical sites that required urgent medical intervention, (e.g., CNS metastasis with potential for spinal cord progression).
Two or more consecutive doses of study therapy were missed due to non-compliance, unless otherwise approved by Sponsor.
Pregnancy

Tumor flare phenomenon, defined as local pain, irritation, or rash localized at sites of known or suspected tumor, did not require treatment discontinuation.

Administration of regorafenib or trifluridine and tipiracil was permanently discontinued if a patient had confirmed disease progression or unacceptable toxicity. Refer to the approved prescribing information for additional information regarding the discontinuation of regorafenib or trifluridine and tipiracil.

### Definition of End of Study

If the study was not terminated for a reason provided above, the overall study ended 24 months after the last patient completed the 90-day safety follow-up visit; if the last patient withdrew from the study, or was lost to follow-up (i.e., the patient was unable to be contacted by the Investigator) prior to the 90-day safety follow-up visit, the study would have ended approximately 24 months from the date of that event.

B. Study population

### Inclusion Criteria

In order to participate in the study, a patient must have met all of the following inclusion criteria:
1. Histologically confirmed diagnosis of unresectable and metastatic colorectal adenocarcinoma.
2. The tumor must have been assessed for MSI-H or dMMR status per a standard local testing method.
3. Patient, or Legally Authorized Representative if patient is unable to do so, voluntarily agreed to participate by giving signed, dated, and written informed consent prior to any study-specific procedures.
4. ≥18 years of age.
5. Must have received at least 1 prior chemotherapy for metastatic or recurrent CRC as follows:
   a. Standard chemotherapy including all of the following agents (if eligible and no contraindication): a fluoropyrimidine, irinotecan, oxaliplatin, and an anti-EGFR antibody (cetuximab or panitumumab) if applicable. These agents may have been in combination, e.g., FOLFOXIRI may be given first line in which case a RAS mutant patient may be eligible for this study in the second line, or more commonly, agents will be sequenced, and most patients will be eligible in the third line and beyond.
   b. Patients must have progressed while receiving or within 3 months of the last administration of their last line of standard therapy or be unable to tolerate any of these standard treatments due to toxicity, which warrants discontinuation of treatment and precludes retreatment with the same agent.
   c. Patients who received adjuvant chemotherapy and had recurrence during or within 6 months of completion of the adjuvant chemotherapy can count this as a line of therapy.
6. Measurable disease on baseline imaging per RECIST 1.1.
7. Life expectancy≥12 weeks.
8. ECOG performance status of 0 or 1.
9. Adequate organ function defined as the following laboratory values within 7 days of C1D1:
   a. Neutrophils≥1500/μL (Must have been stable and off any growth factor within 4 weeks of first study treatment administration).
   b. Platelets≥100×103/μL (transfusion to achieve this level was not permitted within 2 weeks of first study treatment administration).
   c. Hemoglobin≥8.0 g/dL (transfusion to achieve this level was not permitted within 2 weeks of first study treatment administration).
   d. Creatinine clearance≥30 mL/min as measured or calculated per local institutional standards.
   e. AST/ALT≤2.5× upper limit of normal (ULN).
   f. Total bilirubin≤1.5×ULN (except patients with Gilbert syndrome who must have a total bilirubin level of ≤3.0×ULN).
   g. Albumin≥3.0 g/dL.
10. The most recent biopsy of a tumor lesion that was available as a formalin-fixed paraffin-embedded (FFPE) tumor tissue block was required. If recent tumor tissue was unavailable or inadequate, patient must have been willing to provide a fresh biopsy if deemed safe and feasible. The sponsor could waive the requirement for screening biopsies once a sufficient number had been collected.
11. Women of childbearing potential (WOCBP) must have had a negative serum pregnancy test at screening (within 72 hours of first dose of study treatment) and prior to study drug administration.
12. Male patients with a female partner(s) of childbearing potential must have agreed to use highly effective contraceptive measures throughout the study starting with the screening visit through 3 months after the last dose of study treatment is received. Males with pregnant partners must have agreed to use a condom; no additional method of contraception was required for the pregnant partner.
13. Willing and able to comply with the requirements of the protocol.

### Exclusion Criteria

Participants were excluded from the trial if any of the following criteria applied:
1. Tumor was MSI-H/dMMR per a standard local testing method
2. Received PD-1, PD-L1, or CTLA-4 antibody including any ICI or experimental immunologic agents.
3. Received regorafenib or trifluridine/tipiracil as prior therapy.
4. Partial or complete bowel obstruction within the prior 3 months, signs/symptoms of bowel obstruction, or known radiologic evidence of impending obstruction.
5. Refractory ascites defined as requiring 2 or more therapeutic paracenteses within the prior 4 weeks or >4 times within the prior 90 days or >1 time within the 2 weeks prior to study entry or requiring diuretics within 2 weeks of study entry.
6. Liver metastases by CT or MRI. NOTE: Patients with definitively treated liver metastases (this includes surgical resection or stereotactic body radiation therapy [SBRT], but not Y-90 or chemotherapy alone) were eligible if they were treated at least 6 months prior to enrollment with no evidence of metastatic disease in the liver on subsequent imaging; however, they must be excluded if they have:
   a. Received>1 SBRT field to the liver.
   b. Undergone major hepatic resection (right, extended right, or extended left) and the remnant liver was subject to SBRT.
   c. Stigmata of hepatic decompensation including a history of variceal bleeding, a history of ascites related to hepatic cirrhosis, or severe portal hypertension.
7. Clinically significant (i.e., active) cardiovascular disease: cerebral vascular accident/stroke or myocardial infarction within 6 months of enrollment, unstable angina, congestive heart failure (New York Heart Association class≥III), or serious uncontrolled cardiac arrhythmia requiring medication.
   a. QTcF (QTc interval corrected using Fridericia's formula) of >480 ms.
8. Active brain metastases or leptomeningeal metastases with the following exceptions:
   a. Treated brain metastases required a) surgical resection, or b) stereotactic radiosurgery. These patients must have discontinued steroid treatment≥10 days prior to randomization for the purpose of managing their brain metastases. Repeat brain imaging following surgical resection or stereotactic radiosurgery was not required if their patient's last brain MRI was within screening window. Whole-brain radiation was not allowed.
   b. Untreated isolated brain metastases that were too small for treatment by surgical resection or stereotactic radiosurgery (e.g., 1-2 mm) and/or of uncertain etiology were potentially eligible but needed to be discussed with and approved by the study Medical Monitor.
9. Concurrent malignancy (present during screening) requiring treatment or history of prior malignancy active within 2 years prior to the first dose of study treatment, i.e., patients with a history of prior malignancy were eligible if treatment was completed at least 2 years before the first dose of study treatment and the patient had no evidence of disease. Patients with history of prior early-stage basal/squamous cell skin cancer, low-risk prostate cancer eligible for active surveillance or noninvasive or in situ cancers who had undergone definitive treatment at any time were also eligible.
10. Treatment with one of the following classes of drugs within the delineated time window prior to C1D1:
   a. Cytotoxic therapy, targeted therapy, or other investigational therapy within 3 weeks.
   b. Monoclonal antibodies, antibody-drug conjugates, radioimmunoconjugates, or similar therapy, within 4 weeks, or 5 half-lives, whichever was shorter.
   c. Small molecule/tyrosine kinase inhibitors within 2 weeks or less than 5 circulating half-lives of investigational drug.
11. Severe acute respiratory syndrome coronavirus 2 (SARS-CoV-2) vaccine or booster<7 days before C1D1.
12. Known allergy or hypersensitivity to any of the study drugs or any of the study drug excipients.
13. Any evidence of current interstitial lung disease (ILD) or pneumonitis, or prior history of ILD or non-infectious pneumonitis requiring glucocorticoids.
14. History of allogeneic organ transplant.
15. Psychiatric or substance abuse disorders that would interfere with cooperation with the requirements of the study.
16. Patients with a condition requiring systemic treatment with either corticosteroids (>10 mg daily prednisone equivalent) within 14 days or another immunosuppressive medication within 30 days of the first dose of study treatment. Inhaled or topical steroids, and adrenal replacement steroid doses (≤10 mg daily prednisone equivalent) were permitted in the absence of active autoimmune disease.
17. Active autoimmune disease or history of autoimmune disease that required systemic treatment within 2 years of the start of study treatment (i.e., with use of disease-modifying agents or immunosuppressive drugs).
18. History or current evidence of any condition, co-morbidity, therapy, any active infections, or laboratory abnormality that might have confounded the results of the study, interfered with the patient's participation for the full duration of the study, or was not in the best interest of the patient to participate, in the opinion of the treating Investigator.
19. Previous SARS-CoV-2 infection within 10 days for mild or asymptomatic infections or 20 days for severe/critical illness prior to C1D1.
20. Uncontrolled infection with human immunodeficiency virus (HIV). Patients on stable highly active antiretroviral therapy with undetectable viral load and normal CD4 counts for at least 6 months prior to study entry were eligible. Serological testing for HIV at screening was not required.
21. Known to be positive for hepatitis B virus (HBV) surface antigen, or any other positive test for HBV indicating acute or chronic infection. Patients who were receiving or who had received anti-HBV therapy and had undetectable HBV DNA for at least 6 months prior to study entry were eligible. Serological testing for HBV at screening was not required.
22. Known active hepatitis C virus (HCV) as determined by positive serology and confirmed by PCR. Patients on or who had received antiretroviral therapy were eligible provided they were virus-free by PCR for at least 6 months prior to study entry. Serological testing for HCV at screening was not required.
23. Had urine protein≥1 gram/24 hour.
24. Uncontrolled hypertension: systolic pressure≥150 mmHg or diastolic pressure≥90 mmHg on repeated measurements that could not be managed by standard antihypertension medications≤28 days before the first dose of study drug(s).
25. Patients who required treatment with strong CYP3A4 inducers or inhibitors.
26. Had presence of gastrointestinal condition, e.g., malabsorption, that might have affected the absorption of study drug(s).
27. Non-healing wound(s).
28. Symptomatic active bleeding.

### C. Study Assessments and Procedures

All screening evaluations were completed and reviewed to confirm that potential patients met all eligibility criteria. The Investigator maintained a screening log to record details of all patients screened and to confirm eligibility or record reasons for screening failure, as applicable.

Procedures conducted as part of the patient's routine clinical management (e.g., blood count) and obtained before signing of the informed consent form (ICF) could be utilized for screening or baseline purposes provided the procedure met the protocol-specified criteria and were performed within the time frame of other baseline assessments.

Repeat or unscheduled samples and images could be taken for safety reasons or for technical issues with the samples.

### Screening

The Investigator, or qualified designee, obtained documented informed consent from each potential patient prior to participating in the clinical study. After a patient signed an ICF, the patient was assigned a unique, sequential patient number. Once a number was assigned, it could not be reassigned if the original patient was found to be ineligible or withdrew consent.

Patients who fulfilled all the inclusion criteria and none of the exclusion criteria were enrolled into the study. Patients who did not meet the inclusion and exclusion criteria were considered screen failures, and their demographic information and reason for screen failure were documented.

During the screening period, attention was given to washout periods for prior treatments and prohibited medications.

### Treatment and Evaluation Period

Patients continued to receive all assessments while they were actively receiving treatment.

### Treatment Discontinuation Visit

The discontinuation visit (≤7 days after meeting criteria for permanent discontinuation) occurred only if study drug was discontinued for any reason other than completion of full study therapy duration. If the discontinuation visit occurred approximately 30 days from the last dose of study drug, at the time of the mandatory 30-Day Safety Follow-Up Visit, procedures did not need to be repeated. If a patient was discontinued from study treatment at a clinic visit, Treatment Discontinuation Visit assessments were performed; the patient did not need to return for a separate Treatment Discontinuation Visit.

### 30-Day Safety Follow-Up Visit

The mandatory 30-Day Safety Follow-up Visit was conducted for all patients 30 days (±7 days) after the last dose of study drug or before initiation of a new antineoplastic treatment, whichever came first. Patients with an AE of Grade>1 were further followed until the resolution of the AE to Grade 0 to 1 or until initiation of a new antineoplastic therapy, whichever occurred first. Patients with a TRAE ongoing at the safety visit were followed until the TRAE resolved, became stable, or was considered not clinically significant by the Investigator.

All patients who discontinued treatment for any reason also had a 90-Day Safety Follow-up Visit (±7 days).

### Efficacy Follow-Up (Off Treatment, on Study)

Patients who discontinued study treatment for reasons other than PD continued with imaging assessments on schedule.

### Survival Follow-Up

For patients who were no longer continuing with study visits and imaging (e.g., after starting subsequent anti-neoplastic therapy), the patient was contacted by telephone Q12W (±14 days) to assess survival status until the overall study ended. Survival data were obtained from public records for patients who have been lost to follow-up.

### Post Study Anti-Cancer Therapy

The Investigator, or qualified designee, reviewed all new anti-cancer therapy initiated after the last dose of study drug. If a patient initiated a new anti-cancer therapy within 4 weeks after the last dose of study drug, the 30-Day Safety Follow-up Visit must have occurred before the first dose of the new therapy. Once new anti-cancer therapy had been initiated, patients were moved into Survival Follow-up.

### Efficacy Assessments

Response assessment was performed according to RECIST 1.1 (Eisenhauer 2009). For all patients, tumor response assessment was obtained by radiographic imaging with CT (preferred) or MRI (if CT was contraindicated) evaluation of the C/A/P (plus other regions as required for specific tumor types or disease history). In general, lesions detected at baseline were followed using the same imaging methodology and preferably the same imaging equipment at subsequent tumor evaluation visits. During disease evaluation, if a lesion was thought to represent fibrosis or scarring, the site could use a fine needle aspiration biopsy or a fluorodeoxyglucose-PET to investigate if residual disease was present. If no active disease was present, that patient could be considered a CR as per RECIST 1.1. Tumor responses to treatment were assigned based on evaluation of response of target, nontarget, and new lesions according to RECIST 1.1 (all measurements were recorded in metric notation) (Eisenhauer 2009). To assess objective response, tumor burden at baseline was estimated and used for comparison with subsequent measurements. At baseline, tumor lesions were categorized in target and non-target lesions (Eisenhauer 2009). Results for these evaluations were recorded with as much specificity as possible so that pre- and post-treatment results provide the best opportunity for accurately evaluating tumor response.

Any CR or PR was confirmed at the next scheduled scan (Eisenhauer 2009). If a patient discontinued treatment but remained on study, scans were taken according to the treatment schedule. Additional imaging was performed if clinically indicated per the Investigator's discretion.

### Tumor Imaging

Initial tumor imaging was performed during the screening period to establish a baseline of disease burden. Scans performed as part of routine clinical management were acceptable for use as a screening scan if they were of adequate quality and ≤21 days prior to the first dose.

The Investigator could perform scans in addition to a scheduled study scan if clinically indicated per the Investigator's discretion. The timing of on-study imaging followed calendar days and was not adjusted for delays in treatment administration or for visits. The same imaging technique was used in a patient throughout the study for consistency.

### Brain Imaging

MRI, with and without contrast, was the preferred brain imaging modality; however, CT was acceptable if MRI was clinically contraindicated. Patients with a history of CNS metastases received brain imaging on the same schedule as the chest/abdomen/pelvis imaging.

During the study, brain CT/MRI scans were conducted if clinically indicated by development of new symptoms.

### Assessment Of Safety

The safety profile of the study treatment was assessed through the recording, reporting, and analyzing of baseline medical conditions, AEs, physical examination findings, including vital signs, and laboratory tests. Comprehensive assessment of any apparent toxicity experienced by the patient was performed throughout the course of the study, from the time of the patient's signature of informed consent. Study site personnel reported any AE, whether observed by the Investigator or reported by the patient

### Primary Efficacy Analyses

### Objective Response Rate (ORR)

ORR is defined as the proportion of patients who had best overall response (BOR) of objective responses (CR or PR). BOR is defined as the best response recorded from randomization until data cutoff, disease progression, or the start of new anti-cancer treatment. Patients with no post-baseline response assessment were considered as non-responders for BOR. Confirmed ORR assessed by the investigator per RECIST 1.1 was reported in each arm, as well as its corresponding Clopper-Pearson 95% CI. In addition, the ORR difference between arms was calculated with 95% CI constructed using Miettinen Nurminen method (Miettinen 1985). There were two types of comparisons: 1) comparison of the lower dose arms versus the higher dose arms (combination: Arm A vs. Arm B and monotherapy: Arm C vs. Arm D), and 2) comparison of combination treatment arms versus monotherapy arms (lower dose: Arm A vs. Arm C and higher dose: Arm B vs. Arm D). The analyses also included comparisons to Arm E to better demonstrate the contribution of component and to characterize the treatment effects of botensilimab with or without balstilimab. The study was not powered for hypothesis testing. Therefore, any lack of statistical significance should not be interpreted as evidence of no difference. Subgroup analysis was performed on the primary efficacy endpoint in ITT Analysis Set.

The primary analysis was performed approximately 6 months after the last patient was randomized, and it is considered as the final analysis of the study. The supplemental analysis including updated efficacy and safety data was performed upon completion of the study.

### Secondary Efficacy Analyses

### Duration of Response (DOR)

DOR is defined as the time from initial objective response until the first documentation of progression assessed by investigator per RECIST 1.1 or death, whichever came first. DOR was summarized using the Kaplan-Meier method in the responders only. All the censoring rules for PFS analysis were applied to DOR as well. The median DOR and its 95% CI, where estimable, was constructed with generalized Brookmeyer and Crowley method (Brookmeyer 1982). The cumulative probability of DOR at 3-month intervals was calculated and presented with a two-sided 95% CI by using Greenwood's formula.

### Progression-Free Survival (PFS)

PFS is defined as time from randomization until progression assessed by investigator per RECIST 1.1 or death, whichever came first. Patients without an event (death or PD) at the analysis cutoff date were censored on the date of last tumor assessment or start of new anti-cancer therapy. The median PFS and the cumulative probability of PFS at 3-month intervals was calculated using Kaplan-Meier method for each treatment arm and presented with a two-sided 95% CI. The PFS censoring rule followed the FDA Guidance for Industry Clinical Trial Endpoints for the Approval of Cancer Drugs and Biologics (Food and Drug Administration 2007). Data for patients without disease progression or death at the time of analysis was censored at the time of the last adequate tumor assessment. Data for patients who were lost to follow-up prior to documented disease progression were censored at the last adequate tumor assessment date when the patient was known to be progression free. Data for patients who started to receive new anti-cancer therapy were censored at the last adequate tumor assessment date prior to the introduction of new therapy.

At final analysis, PFS distribution between treatment arm was compared descriptively in a log-rank test. The hazard ratio between arms were estimated from the cox regression model.

### Overall Survival (OS)

OS, defined as time to death of any cause, was analyzed in the ITT Analysis Set; patients were censored either at the date that the patient was last known to be alive or the date of data cutoff, whichever came earlier. The median OS and cumulative probability of OS estimated at 6-month intervals was calculated using Kaplan-Meier estimates for each treatment arm and presented with a two-sided 95% CI. Descriptive comparison of OS between arms was made similarly as in PFS at the final analysis.

### Safety Analyses

All safety endpoints were analyzed in the Safety Analysis Set, using actual treatment assignments.

### Extent of Exposure

Extent of exposure to each study drug was summarized descriptively as the number of doses received (number and percentage of patients), duration of exposure (days), cumulative total dose received per patient (mg), dose intensity, and relative dose intensity. The number (percentage) of patients requiring dose interruption, dose delays, and drug discontinuation due to AEs was summarized for each study drug. Frequency of the above dose adjustments and discontinuation was summarized by category.

Patient data listings were provided for all dosing records and for calculated summary statistics.

### Adverse Events (AEs)

All AEs were coded in MedDRA v25.0 or higher and graded by NCJ CTCAE v5.0. AEs that had an onset date or a worsening in severity from baseline (pre-treatment) on or after the first dose of study drug and up to 90 days following discontinuation of the study treatment (i.e., the last dose of randomized treatment) or until the initiation of the first subsequent anti-cancer therapy (including radiotherapy, with the exception of palliative radiotherapy) following discontinuation of study treatment (whichever occurred first) were considered a treatment-emergent adverse event (TEAE) and included in summary tables. All AEs, treatment-emergent or not, were included in the listings.

The incidence of TEAEs was reported as the number (percentage) of patients with TEAEs by system, organ, class, and preferred term. The number (percentage) of patients with TEAEs was also summarized by relationship to the study drug. TRAEs include those AEs considered by the investigator to be related to a study drug or with missing assessment of the causal relationship.

SAEs, deaths, TEAEs with Grade≥3 severity, irAEs, TRAEs and TEAEs leading to treatment discontinuation, dose interruption, or dose delay were summarized.

### D. Pharmacokinetics

Serum botensilimab and balstilimab PK parameters include (but are not limited to) Cₘₐₓ₋ₛₛ, Cₘᵢₙ₋ₛₛ, area under the drug concentration-time curve within time span t1 to t2 at steady state (AUC₍ₜ₁₋ₜ₂₎₋ₛₛ), area under the drug concentration-time curve from time zero to time t (AUC₍₀₋ₜ₎), area under the drug concentration-time curve from time zero to infinity (AUC_{(0-∞)}), tₘₐₓ, terminal elimination rate constant (λz), t_{1/2}, systemic drug clearance (CL), and Vd.

Both noncompartmental and compartmental modeling (e.g., PopPK) may be used to analyze PK. Additional PK exposure metrics for serum botensilimab and balstilimab were assessed via PopPK.

### E. Immunogenicity

The immunogenicity assessment was conducted to detect and measure ADA (antibodies against botensilimab and balstilimab) using multi-tiered strategy of screening, confirmatory, and titer assays. The samples that were positive in ADA assay were tested for neutralizing antibodies. Blood samples for serum botensilimab and balstilimab immunogenicity/ADA were collected from patients at the designated timepoints. Blood samples may be used for additional bioanalytical characterization.

### F. Tumor Biomarkers

Tumor tissue was collected during the study. Tumor tissue biomarker measurements include but are not limited to the following: PD-L1 expression, microsatellite instability status, and other markers as deemed relevant for the current study.

### G. Objectives and Endpoints

TABLE 5
Objectives and endpoints

| | |
|---|---|
| Objectives | Endpoints |

Primary

| | |
|---|---|
| To evaluate the clinical | ORR, defined as the proportion of |
| efficacy of botensilimab | patients with a CR or PR as |
| as monotherapy and in | assessed by RECIST 1.1 criteria. |
| combination with balstilimab | |
| through ORR as assessed by | |
| the investigator per RECIST | |
| 1.1 in the Intent-to-Treat | |
| (ITT) Analysis Set | |

Secondary

| | |
|---|---|
| To evaluate the clinical | DOR, defined as the time from |
| efficacy of botensilimab | initial objective radiographic |
| as monotherapy and in | response until disease progression |
| combination with balstilimab | or death, whichever occurred first. |
| as determined by DOR in the | PFS, defined as the time from |
| ITT Analysis Set. | randomization until disease |
| To evaluate the clinical | progression or death, whichever |
| efficacy of botensilimab | occurred first. |
| as monotherapy and in | OS, defined as the time from |
| combination with an | randomization until death |
| balstilimab as determined by | due to any cause |
| progression-free survival | |
| (PFS) in the ITT Analysis Set. | |
| To evaluate the clinical | |
| efficacy of botensilimab | |
| as monotherapy and in | |
| combination with balstilimab | |
| as determined by OS in the | |
| ITT Analysis Set | |

Safety

| | |
|---|---|
| To evaluate the safety and | AEs, serious adverse events |
| tolerability of botensilimab | (SAEs), AEs leading to treatment |
| as monotherapy and in | discontinuation or death. |
| combination with balstilimab | |
| in the Safety Analysis Set. | |

Pharmacokinetics/Immunogenecity

| | |
|---|---|
| To further characterize the PK | Serum botensilimab ± balstilimab |
| profile and immunogenicity of: | PK exposure parameters. |
| botensilimab as monotherapy. | Serum botensilimab ± balstilimab |
| botensilimab and balstilimab | ADAs. |
| in combination. | |

The primary analysis was performed approximately 6 months after the last patient was randomized; it is considered as the final analysis of the study. The supplemental analysis including updated efficacy and safety data was performed upon completion of study.

### H. Initial Results

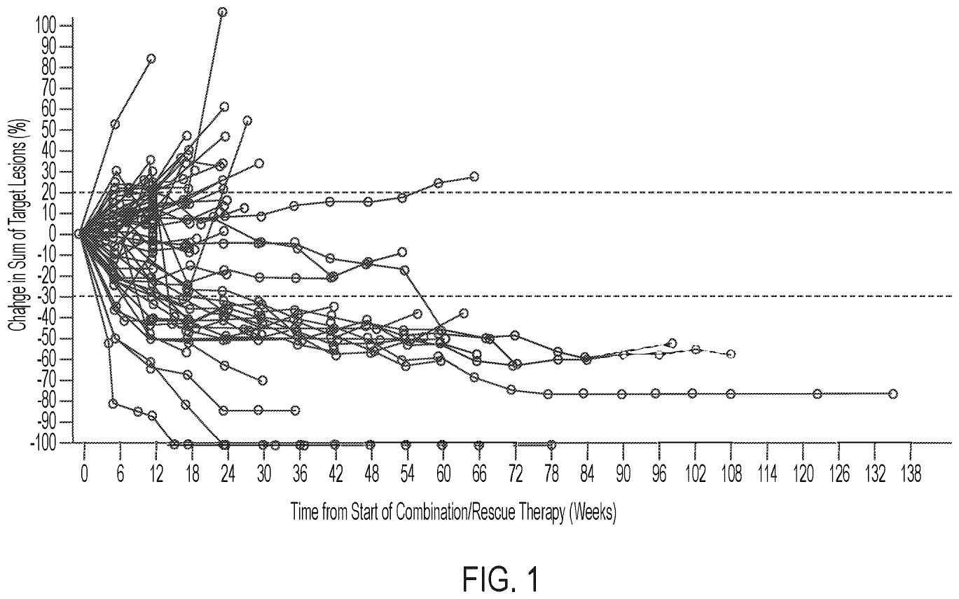

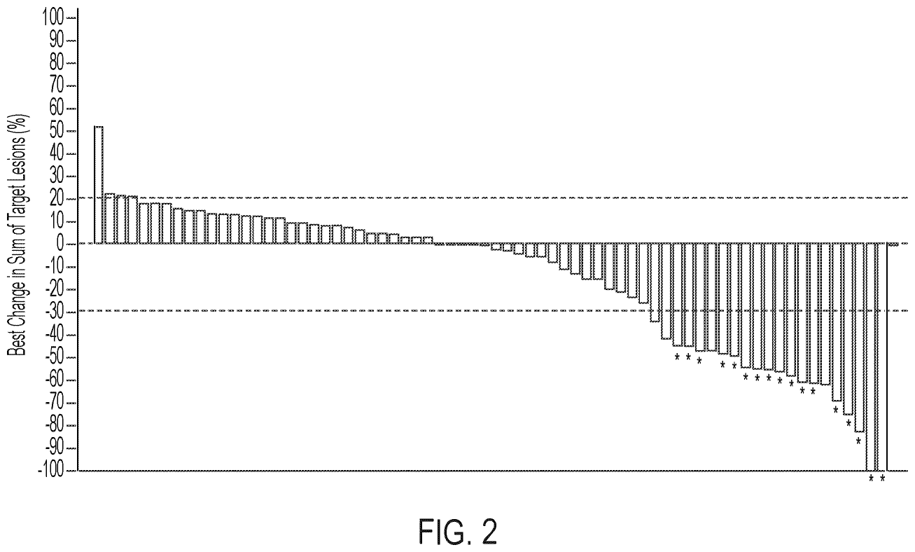

Initial analysis was performed to evaluate the best overall response (BOR), objective response rate (ORR), and disease control rate (DCR), in patient groups with no active liver metastases, including an efficacy evaluable (EE) population group (n=70) and an intent to treat (ITT) population group (n=77). A total of 70 efficacy evaluable patients received botensilimab (1 mg/kg or 2 mg/kg every six weeks) and balstilimab (3 mg/kg every two weeks). The change in sum of target lesions from the start of combination/rescue therapy in the EE population group is shown in FIG. 1, and the best change in sum of target lesions is shown in FIG. 2. BOR evaluation of patients in the EE population group showed 1 patient (1%) with complete response (CR); 16 patients (23%) with partial response (PR); 39 patients (56%) with stable disease (SD); and 14 patients (20%) with progressive disease (PD). The DCR for the EE population group, defined as CR+PR+SD, was 80%, with a 95% confidence interval of 69% to 89%. The confirmed ORR for the EE population group was 24%, with a 95% confidence interval of 15% to 36%. The median follow-up time was 12.3 months, with a range of 1.4 months to 40.5 months, and there were 10 responses ongoing (59%). In the ITT population group, the ORR was 22%, and the DCR was 73%. The reported ORR for patients treated via the current standard of care was 2.8%.

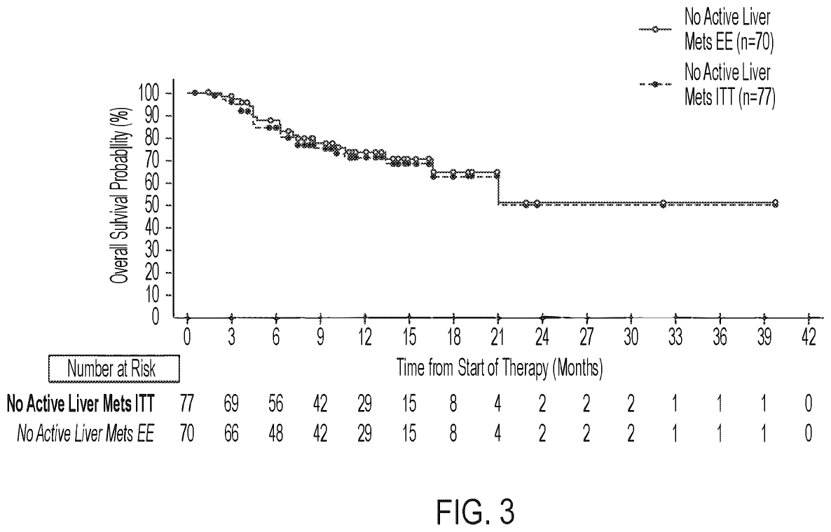

Current overall survival probability was also analyzed for the EE and ITT population groups, as shown in FIG. 3 and summarized in Table 6, below. Further analysis of the study results is ongoing.

TABLE 6
Current survival probabilities in EE and ITT population groups.

| | | | Median |
|---|---|---|---|
| | | 12-month | follow-up |
| | Median OS, | OS, % | time, months |
| | months (95% CI) | (95% CI) | (range) |
| EE population | Not reached (16.5, | 74% | 12.3 |
| group | not reached) | (61%, 83%) | (1.4, 40.5) |
| ITT population | Not reached (16.5, | 71% | 11.1 |
| group | not reached) | (58%, 81%) | (0.6, 40.5) |

### INCORPORATION BY REFERENCE

All patent and non-patent literature references cited above are incorporated herein by reference in their entirety.

1. A method of treating colorectal cancer in a subject in need thereof, the method comprising administering to the subject an antibody that specifically binds to human Cytotoxic T-Lymphocyte Antigen 4 (CTLA-4) at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

2. A method of enhancing the activation of T cells colorectal cancer in a subject who has colorectal cancer, the method comprising administering to the subject an antibody that specifically binds to human CTLA-4 at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

3. The method of claim 1, wherein the antibody is administered at a dose of 50 mg to 175 mg.

4. The method of claim 1, wherein the antibody is administered at a dose of 75 mg to 150 mg.

5. The method of claim 1, wherein the antibody is administered at a dose of 25 mg, 50 mg, 75 mg, 100 mg, or 150 mg.

6. The method of claim 1, wherein the antibody is administered intravenously.

7. The method of claim 1, wherein the antibody is administered by intravenous infusion over about 30 minutes.

8. The method of claim 1, wherein the antibody is administered once weekly.

9. The method of claim 1, wherein the antibody is administered once every 2 weeks.

10. The method of claim 1, wherein the antibody is administered once every 3 weeks.

11. The method of claim 1, wherein the antibody is administered once every 4 weeks.

12. The method of claim 1, wherein the antibody is administered once every 5 weeks.

13. The method of claim 1, wherein the antibody is administered once every 6 weeks.

14. The method of claim 1, wherein the antibody is administered intravenously at a dose of 25 mg once every 6 weeks.

15. The method of claim 1, wherein the antibody is administered intravenously at a dose of 50 mg once every 6 weeks.

16. The method of claim 1, wherein the antibody is administered intravenously at a dose of 75 mg once every 6 weeks.

17. The method of claim 1, wherein the antibody is administered intravenously at a dose of 100 mg once every 6 weeks.

18. The method of claim 1, wherein the antibody is administered intravenously at a dose of 150 mg once every 6 weeks.

19. The method of claim 1, wherein the dose is a therapeutically effective amount.

20. The method of claim 1, wherein the colorectal cancer is colorectal adenocarcinoma.

21. The method of claim 1, wherein the colorectal cancer is unresectable.

22. The method of claim 1, wherein the antibody is administered to the subject prior to surgical resection of a primary tumor.

23. The method of claim 1, wherein the colorectal cancer is metastatic.

24. The method of claim 1, wherein the subject does not have liver metastases.

25. The method of claim 1, wherein the colorectal cancer is relapsed and/or refractory.

26. The method of claim 1, wherein the subject has not received any prior chemotherapy.

27. The method of claim 1, wherein the subject has not received any prior radiation therapy.

28. The method of claim 1, wherein the subject has received at least one prior chemotherapy.

29. The method of claim 28, wherein the at least one prior chemotherapy is fluoropyrimidine, irinotecan, oxaliplatin, or an anti-EGFR antibody.

30. The method of claim 29, wherein the anti-EGFR antibody is cetuximab or panitumumab.

31. The method of claim 1, wherein the subject has previously been treated with folinic acid, 5-fluorouracil, oxaliplatin, and irinotecan.

32. The method of claim 1, wherein the subject is unable to tolerate a standard of care treatment.

33. The method of claim 1, wherein the subject has a RAS mutation.

34. The method of claim 33, wherein the RAS mutation is a KRAS or NRAS mutation.

35. The method of claim 1, wherein the colorectal cancer is not microsatellite instable—high (MSI-H).

36. The method of claim 1, wherein the colorectal cancer is microsatellite stable (MSS).

37. The method of claim 1, wherein the colorectal cancer is not mismatch repair deficient (dMMR).

38. The method of claim 1, wherein the subject has not previously been treated with an anti-PD-1, anti-PD-L1, or anti-CTLA-4 antibody.

39. The method of claim 1, wherein the subject has not previously been treated with regorafenib, trifluridine, and/or tipiracil.

40. The method of claim 1, wherein the cancer is refractory to a standard of care treatment.

41. The method of claim 40, wherein the standard of care treatment is chemotherapy or radiation.

42. The method of claim 40, wherein the standard of care treatment is folinic acid, 5-fluorouracil, oxaliplatin, irinotecan, fluoropyrimidine, cetuximab, and/or panitumumab.

43. The method of claim 1, wherein administration of the antibody reduces tumor size in the subject.

44. The method of claim 1, wherein administration of the antibody increases T-cell, memory T cell, myeloid cell, and/or antigen presenting cell activation in the subject.

45. The method of claim 1, wherein administration of the antibody reduces the number of Treg cells in the subject.

46. The method of claim 1, wherein before administration of the antibody the subject has measurable disease on baseline imaging per RECIST 1.1.

47. The method of claim 1, wherein before administration of the antibody the subject has an Eastern Cooperative Oncology Group performance status (PS) 0-1.

48. The method of claim 1, wherein before administration of the antibody the subject has a predicted life expectancy of ≥12 weeks.

49. The method of claim 1, wherein before administration of the antibody the subject has:
adequate organ function as defined by one or more of:
a) neutrophils≥1500/μL;
b) platelets≥100×103/μL;
c) hemoglobin≥8.0 g/dL;
d) creatinine clearance≥30 mL/min as measured or calculated per local institutional standards;
e) AST/ALT≤2.5× upper limit of normal (ULN);
f) total bilirubin≤1.5×ULN (except patients with Gilbert syndrome who must have a total bilirubin level of ≤3.0×ULN); and/or
g) albumin≥3.0 g/dL.

50. The method of claim 1, wherein the subject does not have partial or complete bowel obstruction within the last 3 months, signs/symptoms of bowel obstruction, or known radiologic evidence of impending obstruction.

51. The method of claim 1, wherein the subject does not have refractory ascites defined as requiring 2 or more therapeutic paracenteses within the last 4 weeks or ≥4 times within the last 90 days or ≥1 time within the last 2 weeks prior to administration of the antibody.

52. The method of claim 1, wherein the subject does not have clinically significant cardiovascular disease.

53. The method of claim 1, wherein the subject does not have active brain metastases or leptomeningeal metastases.

54. The method of claim 1, wherein the subject does not have a concurrent malignancy that requires treatment or a history of prior malignancy that was active within 2 years prior to administration of the antibody.

55. The method of claim 1, wherein the subject has not had a cytotoxic therapy or targeted therapy, within 3 weeks prior to administration of the antibody.

56. The method of claim 1, wherein the subject has not had other monoclonal antibody therapy, antibody-drug conjugate therapy, or radioimmunoconjugate therapy, within 4 weeks prior to administration of the antibody.

57. The method of claim 1, wherein the subject has not had small molecule tyrosine kinase inhibitor therapy within 2 weeks prior to administration of the antibody.

58. The method of claim 1, wherein the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

59. The method of claim 1, wherein the antibody comprises a VH comprising the amino acid sequence set forth in SEQ ID NO: 7 and a VL comprising the amino acid sequence set forth in SEQ ID NO: 8.

60. The method of claim 1, wherein the antibody comprises a human IgG1 heavy chain constant region comprising S239D/A330L/I332E mutations, numbered according to the EU numbering system.

61. The method of claim 1, wherein the antibody comprises a heavy chain comprising the amino acid sequence set forth in SEQ ID NO: 9 and a light chain comprising the amino acid sequence set forth in SEQ ID NO: 10.

62. The method of claim 1, wherein the antibody is botensilimab.

63. The method of claim 1, wherein the method further comprises administering an antibody that specifically binds to human PD-1 to the subject.

64. The method of claim 63, wherein the antibody that specifically binds to human PD-1 comprises: a heavy chain variable region (VH) comprising the CDRH1, CDRH2, and CDRH3 amino acid sequences of the VH amino acid sequence set forth in SEQ ID NO: 17; and a light chain variable region (VL) comprising the CDRL1, CDRL2, and CDRL3 amino acid sequences of the VL amino acid sequence set forth in SEQ ID NO: 18.

65. The method of claim 63, wherein the antibody that specifically binds to human PD-1 comprises the CDRH1, CDRH2, CDRH3, CDRL1, CDRL2, and CDRL3 amino acid sequences set forth in SEQ ID NOs: 11, 12, 13, 14, 15, and 16, respectively.

66. The method of claim 63, wherein the antibody that specifically binds to human PD-1 comprises a VH comprising the amino acid sequence set forth in SEQ ID NO: 17 and a VL comprising the amino acid sequence set forth in SEQ ID NO: 18.

67. The method of claim 63, wherein the antibody that specifically binds to human PD-1 comprises a heavy chain comprising the amino acid sequence set forth in SEQ ID NO: 19 and a light chain comprising the amino acid sequence set forth in SEQ ID NO: 20.

68. The method of claim 63, wherein the antibody that specifically binds to human PD-1 is balstilimab.

69. The method of claim 63, wherein the antibody that specifically binds to human PD-1 is administered at a dose of 200 mg to 300 mg.

70. The method of claim 63, wherein the antibody that specifically binds to human PD-1 is administered at a dose of 240 mg.

71. The method of claim 63, wherein the antibody that specifically binds to human PD-1 is administered once weekly or once every 2 weeks.

72. The method of claim 63, wherein the antibody that specifically binds to human PD-1 is administered at a dose of 240 mg once every 2 weeks.

73. An antibody that specifically binds to human CTLA-4 for use in the treatment of colorectal cancer, wherein the treatment is performed according to the method of claim 1.

74. An antibody that specifically binds to human CTLA-4 for use in the manufacture of a medicament for the treatment of colorectal cancer, wherein the treatment is performed according to the method of claim 1.

75. Use of an antibody that specifically binds to human CTLA-4 for the treatment of colorectal cancer, wherein the treatment is performed according to the method of claim 1.

76. An antibody that specifically binds to human CTLA-4 and an antibody that specifically binds to human PD-1 for use in the treatment of colorectal cancer, wherein the treatment is performed according to the method of claim 1.

77. An antibody that specifically binds to human CTLA-4 and an antibody that specifically binds to human PD-1 for use in the manufacture of a medicament for the treatment of colorectal cancer, wherein the treatment is performed according to the method of claim 1.

78. Use of an antibody that specifically binds to human CTLA-4 and an antibody that specifically binds to human PD-1 for the treatment of colorectal cancer, wherein the treatment is performed according to the method of claim 1.


SEQUENCE LISTING (20 sequences)

SEQ ID NO: 1 - protein, 5 aa, synthetic construct
SYSMN

SEQ ID NO: 2 - protein, 17 aa, synthetic construct
SISSSSSYIYYAESVKG

SEQ ID NO: 3 - protein, 9 aa, synthetic construct
VGLFGPFDI

SEQ ID NO: 4 - protein, 11 aa, synthetic construct
RASQSVSRYLG

SEQ ID NO: 5 - protein, 7 aa, synthetic construct
GASTRAT

SEQ ID NO: 6 - protein, 9 aa, synthetic construct
QQYGSSPWT

SEQ ID NO: 7 - protein, 118 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 8 - protein, 107 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 9 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYGMH

SEQ ID NO: 12 - protein, 17 aa, synthetic construct
VIWYDGSNKYYADSVKG

SEQ ID NO: 13 - protein, 4 aa, synthetic construct
NGDH

SEQ ID NO: 14 - protein, 11 aa, synthetic construct
RASQSVSSNLA

SEQ ID NO: 15 - protein, 7 aa, synthetic construct
GASTRAT

SEQ ID NO: 16 - protein, 9 aa, synthetic construct
QQYNNWPRT

SEQ ID NO: 17 - protein, 113 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSS

SEQ ID NO: 18 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 19 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC